(19) United States
(12) Patent Application Publication
(10) Pub. No.: US 2004/0170591 A1
(43) Pub. Date: Sep. 2, 2004

(54) Dermatological composition comprising nicotinic acid or an amide, and a sphingoid base

(76) Inventors: Jean-Claude Allart, Labuissiere (FR); Jean-Marie Lefevre, Amiens (FR); Jacques Peyrot, Clermont Ferrand (FR)

Correspondence Address: E Joseph Gess, Burns Doane Swecker & Mathis, P O Box 1404, Alexandria, VA, 22313-1404, US

(21) Appl. No.: 10/475,648

(22) PCT Filed: Apr. 17, 2002
(86) PCT No.: PCT/FR02/01330

(30) Foreign Application Priority Data
Apr. 23, 2001 (FR) 01/05451

(51) Int. Cl. A61K 31/455; A61K 7/075

(52) U.S. Cl. 424/70.21; 514/355; 514/356

(57) ABSTRACT

The invention concerns a dermatological composition comprising in combination at least nicotinic acid or a nicotinic acid amide such as nicotinamide (vitamin PP), and at least a sphingoid base selected among sphingosine, sphinganine, phytosph-ingosine, tetracetylphytosphingosine, N-acetylphytosphingosine, and phytosphingosine hydrochloride. The invention is useful for treating atopic dermatitis and acne.

The present invention relates to a composition comprising essentially nicotinic acid or an amide of nicotinic acid and a sphingoid base, which is useful in dermatology, and more particularly to a dermatological composition comprising, in combination, at least nicotinic acid or an amide of nicotinic acid and at least one sphingoid base, for treating keratinization disorders in human or animal dermatology.

The skin is a true organ, having properties which are essential to life. It comprises superficial layers, namely the epidermis, and deeper layers, the dermis and the hypodermis, and each one has specific properties enabling the whole to react and adapt to the conditions of its environment. It plays a protective role with respect to the environment, and also an immune role, the correct functioning of which conditions the well-being of the individual. Any problem with its physiology has pathological consequences which are of varying seriousness but which always justify rapid and appropriate treatment. This shows the importance of understanding properly the mechanisms which are involved in being able to provide a judicious therapeutic response with no risk of worsening the situation.

Acne and atopic dermatitis are epidermal conditions which affect a large number of individuals, including children and adolescents. Atopic dermatitis is a common condition affecting individuals of both sexes from the age of three months, characterized by repeat attacks of eczema on skin exhibiting abnormalities in terms of its constitution, whereas acne is a skin condition associated with abnormalities of the sebaceous glands, affecting mainly adolescents and young adults.

Acne results from occlusion of the upper end and also of the internal portion of the pilosebaceous canal due to abnormal multiplication of keratinocytes, and from the androgenic hormonal hyperactivity often appearing during puberty, which causes a considerable increase in seborrhea in the sebaceous glands. The blocking of the pilosebaceous canal causes the formation of comedones or microcysts, accompanied by proliferation of Propionibacterium acnes bacteria and of Pytirosporum ovale in the blocked pilosebaceous follicles.

This condition, which is particularly common in adolescents, is accompanied by an inflammatory reaction of the skin, which can be in the form of papules or pustules generally located in the superficial dermis. In certain cases, the inflammatory reaction can reach the deep dermis, forming nodules and macrocysts.

Conventional treatments for acne use benzoyl peroxide, erythromycin, isotretinoin and various antiseptics. However, the use of antibiotics such as erythromycin is today confronted with phenomena of bacterial resistance and of floral modifications, while benzoyl peroxide causes skin irritations and allergies.

Atopic dermatitis is a chronic inflammation of the skin, generally associated with a keratinization problem of origin, with the presence of bacterial overpopulation (Staphylococcus aureus) or yeast overpopulation (Pytirosporum ovale) at the skin surface, the immunogenic role of which explains the existence of a latent inflammatory state, or else with abnormalities of the immune reactions related to an increased penetration of potential allergens from the environment and a disturbed lymphocyte response.

The aim of treatments generally proposed for atopic dermatitis is to act on the various parameters which trigger this condition. Thus, the use of antiseptics to reduce the bacterial overpopulation is increasingly contested in the medical profession since it fragilizes the skin and selects certain microorganisms, conventional emollients are sometimes poorly tolerated by patients, antihistamines are generally ineffective, corticosteroids applied locally have the disadvantage of inducing dermo-epidermal atrophy and a risk of systemic effect, and cyclosporin can cause renal failure. Recent studies have shown that some immune response-modulating agents with anti-inflammatory activity, such as ascomycin and derivatives, could be used in the treatment of atopic dermatitis [K. Rappersberger et al., “Clearing of psoriasis by a novel immunosuppressive macrolide” J. Invest. Dermatol. (1996) 106, 701-710].

The properties of nicotinic acid and of nicotinamide are well known in therapeutics and in dermatology. These vitamins are, for example, useful for treating acne, in particular common acne, and patent EP-B-052 705 describes various liquid and solid forms intended for their use in these indications. However, it is known that nicotinic acid and nicotinamide (vitamin PP) used on their own do not provide a satisfactory therapeutic efficacy in the treatment of acne or of atopic dermatitis.

Patent FR-A-2 780 888 describes a lotion intended for the dermatological treatment of comedones comprising essentially a combination of nicotinamide (vitamin PP) and hydroxy acids, more particularly mandelic acid. Some nicotinic acid derivatives, and in particular vitamin B3, have been proposed in combination with ceramide precursors in application WO 99/47114, but the compositions containing them are intended for the moisturization of normal skin and not for dermatological treatments of pathological skin.

It is known that sphingoid bases, such as phyto-sphingosine and sphingosine, which are ceramide precursors, are present in human skin, and studies have shown that these molecules have protein kinase C-inhibiting properties and appear to be involved in the differentiation of epidermal keratinocytes. It has also been observed that sphingosines present in the stratum corneum and other layers of the epidermis inhibit the growth of certain undesirable microorganisms.

Various publications describe the use of sphingosine and of phytosphingosine in cosmetic and dermatological compositions. For example, patent application WO 95/03028 describes sphingosine, used in a composition at a pH in the region of 5, to reduce the irritant effect of certain alpha-hydroxy acids. Patent EP 940 140 describes a cosmetic composition which combines alpha-hydroxy acids and ceramides or ceramide precursors such as phytosphingosine. However, to date, sphingoid bases have not proved to be effective in the treatment of conditions such as atopic dermatitis and acne.

There remains therefore a need for dermatological compositions with anti-inflammatory activity which allow local treatment of acne and of atopic dermatitis under good conditions in terms of efficacy and of patient comfort, while at the same time avoiding the side effects and the disadvantages of conventional treatments.

The studies carried out by the applicant have shown that it is possible to effectively treat acne and atopic dermatitis by combining nicotinic acid or an amide of nicotinic acid, such as vitamin PP, and a sphingoid base within the same composition.

A subject of the invention is therefore a novel composition which is useful in human and animal dermatology, for the treatment of acne, in particular common acne, and of atopic dermatitis.

A subject of the present invention is also a composition combining nicotinic acid or an amide of nicotinic acid, such as vitamin PP, and a sphingoid base, for the treatment of acne and of atopic dermatitis.

A subject of the invention is also a cosmetological treatment method for the skin, consisting in applying to exposed areas a composition based on nicotinic acid or an amide of nicotinic acid, and a sphingoid base.

Finally, a subject of the present invention is the use of nicotinic acid or an amide of nicotinic acid, such as vitamin PP, and of a sphingoid base, for preparing a medicinal product for the treatment of acne and of atopic dermatitis.

The composition in accordance with the present invention comprises, in combination, at least nicotinic acid or an amide of nicotinic acid and at least one sphingoid base.

The amide of nicotinic acid can preferably be nicotinamide or vitamin PP, and the sphingoid base is chosen from sphingosine, sphinganine, phytosphingosine, tetraacetyl-phytosphingosine, N-acetylphytosphingosine, and phyto-sphingosine hydrochloride. According to the invention, the preferred sphingoid base is phytosphingosine or phytosphingosine hydrochloride.

The nicotinic acid, or the amide of nicotinic acid, is used in the composition in a proportion of 0.1 to 15% by weight relative to the total weight of the composition, and preferably of 1 to 10%. The content of sphingoid base can vary depending on the base used, but it is generally between 0.01% and 5%, preferably between 0.05 and 2%.

According to an advantageous embodiment, the composition of the invention also comprises ciclopirox or ciclopiroxolamine, the action of which effectively completes that of the other two abovementioned components, i.e. the sphingoid base and the nicotinic acid or the amide of nicotinic acid.

Ciclopirox and ciclopiroxolamine can be incorporated into the composition in a proportion of 0 to 5% by weight relative to the total weight of the composition, and preferably between 0.5% and 2% by weight.

The nicotinic acid and the amide of nicotinic acid, in particular vitamin PP, and also the sphingoid bases used in the compositions of the present invention are generally commercially available and can be obtained by known methods from various appropriate sources.

For example, the sphingoid bases can be obtained from natural sources or by chemical synthesis or by fermentation. They can also be obtained from animal or plant tissues by extraction and purification. Preferably, the sphingoid bases used in the invention are prepared by microbial fermentation, for example from a yeast such as Pichia ciferii, and the phytosphingosine obtained in this way has the advantage of being very close to that of human or animal skin. According to a preferred embodiment of the invention, the phytosphingosine obtained from tetraacetylphytosphingosine by deacetylation is used as sphingoid base. The deacetylation reaction can be carried out by chemical reaction, for example by hydrolysis in basic medium, or by enzymatic reaction.

The tests carried out on compositions in accordance with the present invention have demonstrated a synergistic action most particularly between vitamin PP and phytosphingosine, making it possible to act simultaneously on several parameters and providing a potentiation of the anti-acne activity while at the same time eliminating the risk of bacterial resistance and of irritant or allergic phenomena. A single composition according to the present invention can thus be substituted for the combined use of an emollient, an antiseptic and a local anti-inflammatory for the treatment of atopic dermatitis and of acne, which has in particular the advantage of avoiding the side effects of each one of these usual treatment products.

Ciclopirox (international nonproprietary name) and some of its salts, such as ciclopiroxolamine, are compounds known for their antifungal and antibacterial properties allowing them to be used, for example, in antidandruff lotions and shampoos. On the other hand, their use for the treatment of keratinization disorders, such as acne and atopic dermatitis, had not been envisioned up until now.

The addition of ciclopirox or of a salt such as ciclopiroxolamine has the effect of increasing and of accelerating the phenomenon of lysis of yeast strains, in particular Malassezia furfur, whereas the sphingoid base, in particular phytosphingosine, exerts an anti-inflammatory action which limits the cytokine reaction induced by the abrupt lysis that the ciclopirox might trigger. In addition, nicotinic acid or its amides, in particular vitamin PP, promotes the formation of ceramides in the epidermis. Thus, these three categories of active principles effectively combine their activities in the composition according to the invention.

When a composition according to the invention is used in combination with one or more cyclins or isotretinoin, administered orally, it has the advantage of correcting the microorganism selections that these medicinal products can induce, in particular gram-negative forms of folliculitis and staphylococcal forms of folliculitis.

It also makes it possible to regulate the surface lipid film of acne-afflicted skin by enzymatic stimulation of serine palmitoyl transferase and ceramidases, and by activation of fatty acid and cholesterol synthesis. It therefore acts more effectively both on the microorganisms of the acne and on the inflammation that they cause while at the same time contributing to restoring the surface lipids, the abnormalities of which are one of the factors triggering the condition.

The excipients and carriers which can be used in the compositions in accordance with the present invention are those commonly used in preparations for dermatological use, and chosen as a function of the form of administration selected. By way of example, mention may be made of gelling agents, emulsifiers, thickeners, preserving agents, soothing agents, and also washing bases and fragrances.

The emulsifier may be chosen from high molecular weight carboxyvinyl polymers such as Carbopol®, polysorbates such as those marketed under the trademarks Tween 20® or Tween 60®, sorbitan esters and, for example, a sorbitan stearate, palmitate, oleate or laurate (for example Arlacel®). Emulsifiers which may also be used include various derivatives of stearic acid or palmitic acid, such as glyceryl stearate, a propylene glycol stearate, a polyethylene glycol stearate, PEG 100® stearate, a steareth or a ceteareth, or else polyglyceryl-2 sesquioleate, polyoxyethylene cetyl ether, a siloxane polyglucoside, or an emulsifiable silicone.

The gelling agents and thickeners are incorporated into the composition in order to improve the fluidity thereof. They may be chosen, for example, from polyacrylamides of the Carbopol type, acrylate/acrylic acid copolymers and, for example, Aculyn®, cellulose derivatives such as hydroxypropylcellulose and, for example, Klucel®, plant mucopolysaccharides, waxes such as beeswax, clays and natural gums such as xanthan gum.

The soothing agents can be chosen from fatty alcohols and esters, and for example the products based on isostearyl alcohol or on polysaccharidic sorbitol marketed under the trademarks Soothex® and Rhamnosoft®. In general, the usual soothing agents of the art may be suitable in the invention.

The washing base generally consists of surfactants such as ionic, nonionic, cationic or anionic surfactants, for instance betaines, sorbitan esters, and more particularly sodium laureth sulfate or glycol distearate. Use may, for example, be made of the products commercially available under the trademarks Texapon® and Sinnoflor®.

The compositions according to the present invention may be in all the usual pharmaceutical forms suitable for dermatological or cosmetological indication, for topical application.

They may, for example, be in the form of aqueous, oily or aqueous-alcoholic solutions, of dispersions, of sera, of gels (aqueous, anhydrous or lipophilic gels), of micellar lotions, of an aqueous-alcoholic lotion, of solutions for spraying, of suspensions, of ionic or nonionic vesicular dispersions, or of liquid or semi-liquid emulsions (for example a milk) or in solid or in semi-solid form. The emulsions may be of the oil-in-water (O/W) or water-in-oil (W/O) type, for example gels, creams or shampoos.

For the treatment of atopic dermatitis and of acne, the compositions in accordance with the invention are used according to a dosage determined as a function of the seriousness of the condition and of the patient's condition. In general, they are applied 1 to 4 times a day for a period ranging from a few days to a few weeks. In most cases, an application twice a day for 4 to 5 weeks is sufficient for a starting treatment, and the results are already noticeable from the first days.

The following examples illustrate the invention in greater detail without limiting the scope thereof. Unless otherwise indicated, the parts and percentages are expressed by weight.

### EXAMPLE 1

According to the usual techniques, an alcoholic lotion for topical application is prepared, having the composition by weight given below.

| | |
|---|---|
| Phytosphingosine hydrochloride | 0.30 |
| Nicotinamide (vitamin PP) | 4.00 |
| Cyclomethicone | 12.00 |
| C₁₂-C₁₃ alkyl lactate | 14.00 |
| Ethoxy diglycol | 6.00 |
| Ethanol at 96° | q.s. 100.00 |

### EXAMPLE 2

According to the usual techniques, an aqueous-alcoholic gel for topical application is prepared, having the composition by weight given below.

| | |
|---|---|
| Phytosphingosine hydrochloride | 0.15 |
| Nicotinamide (vitamin PP) | 4.00 |
| Cyclomethicone | 12.00 |
| C₁₂-C₁₃ alkyl lactate | 14.00 |
| Ethoxy diglycol | 6.00 |
| Hydroxypropylcellulose | 1.00 |
| Ethanol at 96° | q.s. 100.00 |

### EXAMPLE 3

According to the usual techniques, a micellar lotion is prepared, having the composition by weight given below.

| | |
|---|---|
| Phytosphingosine | 0.20 |
| Nicotinamide (vitamin PP) | 2.00 |
| Tween 80 | 5.00 |
| Laureth-9 | 2.00 |
| Poloxamer-184 | 2.00 |
| Purified water | q.s. 100.00 |

### EXAMPLE 4

An oil-in-water emulsion (cream) is prepared, having the following composition by weight.

| | |
|---|---|
| Phytosphingosine | 0.25 |
| Nicotinamide (vitamin PP) | 3.50 |
| Emulsifier (glyceryl stearate/Ceteareth 20/ | 7.00 |
| Ceteareth 10/cetearyl alcohol/cetyl palmitate) | |
| Cetyl alcohol | 1.00 |
| C₁₂-C₁₃ alkyl lactate | 10.00 |
| Cyclomethicone/dimethiconol | 1.00 |
| Xanthan gum | 0.50 |
| Butylene glycol | 5.00 |
| Preserving agents | 0.50 |
| Purified water | q.s. 100.00 |

### EXAMPLE 5

A shampoo in accordance with the present invention is prepared according to the conventional techniques, and its composition by weight is as follows.

| | |
|---|---|
| Phytosphingosine hydrochloride | 0.10 |
| Nicotinamide (vitamin PP) | 2.00 |
| Emulsifier (Sepiperl N ®) | 2.50 |
| Cyclomethicone | 2.50 |
| Thickener (Aculyn 22/33 ®) | 6.50 |
| Triethanolamine | 1.10 |
| Washing base (surfactants) | 64.00 |
| Preserving agents | 0.20 |
| Purified water | q.s. 100.00 |

### EXAMPLE 6

According to the conventional techniques, a cream (oil-in-water emulsion) is prepared, having the following composition:

| | |
|---|---|
| Phytosphingosine | 0.20 |
| Nicotinamide (vitamin PP) | 3.00 |
| Ciclopirox | 1.00 |
| Emulsifier (glyceryl stearate/Ceteareth 20/ | 7.00 |
| Ceteareth 10/cetearyl alcohol/cetyl palmitate) | |
| Cetyl alcohol | 1.00 |
| C₁₂-C₁₃ alkyl lactate | 10.00 |
| Cyclomethicone | 1.00 |
| Butylene glycol | 5.00 |
| Preserving agents | 0.50 |
| Purified water | q.s. 100.00 |

### EXAMPLE 7

A shampoo having the following composition is prepared by the usual methods of the art:

| | |
|---|---|
| Phytosphingosine (hydrochloride) | 0.20 |
| Nicotinamide (vitamin PP) | 3.50 |
| Ciclopiroxolamine | 1.50 |
| Sodium laureth sulfate | 5.00 |
| Emulsifier (Montanov S) | 2.50 |
| Nonionic surfactant | 5.00 |
| Amphoteric surfactant | 28.00 |
| Thickener (Aculyn) | 4.00 |
| Cyclomethicone | 2.50 |
| Conditioner | 0.80 |
| Triethylamine | 4.00 |
| Preserving agents | 0.20 |
| Demineralized water | q.s. 100.00 |

The pH of the composition is 7.5.

This composition can be used in the form of a shampoo one or more times a week.

### EXAMPLE 8

Clinical trials were carried out on 10 patients (5 boys and 5 girls) 14 to 23 years old, so as to demonstrate the effectiveness of the composition of the invention in the treatment of acne.

The composition used is a gel in accordance with that whose composition is given in example 2.

The 10 patients exhibit inflammatory acne of the face, with the forehead, the cheeks, the nose and the chin being affected. A conventional treatment with benzoyl peroxide (4 cases), with erythromycin (3 cases) and with nicotinamide gel (3 cases) brought no notable improvement.

On the other hand, a notable improvement was observed from the 8th day with a treatment using the gel of example 2, at a rate of two daily applications for a month, under conditions of complete tolerance. The treatment did not cause any drying out, despite the presence of alcohol, and the improvement in the quality of the patients' skin resulted in clearer, smoother skin, with many fewer comedones.

Five other patients exhibiting perioral gram-negative folliculitis induced by taking doxycycline orally were successfully treated with the same gel for three weeks at a rate of two applications a day.

Finally, three patients treated simultaneously with isotretinoin at a dose of 0.5 mg/kg for 5 months and a cream in accordance with the invention, the composition of which is given in example 4, did not exhibit any staphylococcal superinfection throughout the treatment. The inflammatory exacerbations at the beginning of treatment were well controlled and the dryness of the face induced by the retinoid was greatly reduced.

### EXAMPLE 9

Clinical trials were carried out on 10 patients (5 boys and 5 girls) 6 months to 30 years old, so as to demonstrate the effectiveness of the composition of the invention in the treatment of atopic dermatitis.

The composition used is a cream in accordance with that whose composition is given in example 4. It is applied for 6 weeks at a rate of two applications a day.

A significant decrease in the populations of Staphylococcus aureus present on the skin of the patients is then observed. A parallel improvement in the pruritis and in the eczematous manifestations is observed from the second week of treatment, with a very substantial decrease in the need for local corticosteroids, and also a gradual reduction in the dryness of the skin.

These improvements can be explained by the restoring of the barrier effect of the stratum corneum through increased synthesis of ceramides and of other lipids of the intercorneocyte cement under the joint and complementary action of the phytosphingosine and the vitamin PP.

5 other patients, 3 months to 4 years old, suffering from atopic dermatitis on the face were treated by applying a micellar lotion in accordance with example 3 using cotton wool impregnated with the lotion, two to three times a day for 5 weeks.

A substantial and rapid improvement in the patients' condition is then observed, with clearly greater tolerance by comparison with the usual products, and better control of local inflammatory phenomena.

A treatment was performed on 5 other patients suffering from a particular form of atopic dermatitis affecting the scalp, the face and the upper torso, associated with the development of an overpopulation of Pytirosporum responsible for the skin sensitization reaction. The treatment consists in applying jointly, for 4 weeks, a shampoo in accordance with example 5, at a rate of twice a week, and a cream according to example 4, at a rate of two applications daily.

A substantial improvement in the condition of the patients, without side effects, was observed from the second week of treatment.

### EXAMPLE 10

A bacteriological study was carried out as indicated below and demonstrated the inhibitory power of a composition according to the invention, in accordance with that described in example 1, on three bacterial strains, compared with a control, an aqueous-glycolic solution and an aqueous-glycolic solution containing phytosphingosine but not containing vitamin PP.

The bacterial strains used are Propionibacterium acnes, Staphylococcus aureus and Serratia marcescens. The first is usually found in saprophytic pathogenic flora, while the other two are generally present in pathogenic superinfections of the skin. In addition, the trials were also carried out on a strain of yeast, Malassezia furfur, which also forms part of the saprophytic flora of the skin.

The active control used is a solution of quaternary ammonium (Bactopin®).

The aqueous-glycolic control has the following composition:

| | |
|---|---|
| Propylene glycol | 5.0 |
| Ethanol | 54.9 |
| Water | q.s. 100.0 |

The aqueous-glycolic solution containing phytosphingosine has the following composition:

| | |
|---|---|
| Propylene glycol | 5.0 |
| Ethanol | 54.9 |
| Phytosphingosine hydrochloride | 0.2 |
| Water | q.s. 100.0 |

The solution of the invention is a simplified variant of the composition in example 1:

| | |
|---|---|
| Propylene glycol | 5.0 |
| Ethanol | 54.9 |
| Phytosphingosine hydrochloride | 0.2 |
| Vitamin PP | 4.0 |
| Water | q.s. 100.0 |

The compositions above are expressed in parts by weight.

The trials are performed by measuring the area of inhibition of growth of the strains around the wells containing the products to be tested. The larger the area, the greater the inhibitory power of the product and, consequently, the greater its effectiveness on the strains tested. The inhibitory power is zero in the case of the aqueous-glycolic control which contains only propylene glycol, ethanol and water, while it is at a maximum in the case of the active control (solution of quaternary ammonium).

| | | | | Phytosphingosine + |
|---|---|---|---|---|
| Strain | Control | Active control | Phytosphingosine | vit. PP |
| Staphylococcus | 0 | 22 | 17.3 | 22.0 |
| aureus | | | | |
| Serratia | 0 | 18 | 12.3 | 12.3 |
| marcescens | | | | |
| Propionibacterium | 0 | 22 | 10.3 | 15.3 |
| acnes | | | | |
| Malassezia | 0 | 22 | 17.0 | 20.0 |
| furfur | | | | |

It is then noted that the solution in accordance with the invention exhibits an inhibitory power on the four strains, and that its effectiveness is equivalent to that of the aqueous-glycolic control (aqueous-glycolic control) and clearly greater than that of the aqueous-glycolic solution containing phytosphingosine. Furthermore, the antibacterial and anti-yeast activity of the composition of the invention is potentiated by the addition of vitamin PP.

On the other hand, the solution for comparison containing 0.2% phytosphingosine hydrochloride exhibits an antibacterial activity which is less than that of the aqueous-glycolic control.

The active control, consisting of a pure solution of quaternary ammonium, is used in this test as a reference, but it could not be used in this form in dermatology.

1. A dermatological composition which is useful for the treatment of skin conditions, characterized in that it comprises, in combination, at least nicotinic acid or an amide of nicotinic acid, and at least one sphingoid base.

2. The composition as claimed in claim 1, characterized in that the sphingoid base is chosen from sphingosine, sphinganine, phytosphingosine, tetraacetylphytosphingosine, N-acetylphytosphingosine, and phytosphingosine hydrochloride.

3. The composition as claimed in claim 1, characterized in that the amide of nicotinic acid is nicotinamide (vitamin PP).

4. The composition as claimed in either one of claims 1 and 3, characterized in that the content of nicotinic acid, or of amide of nicotinic acid, is between 0.1 and 15% by weight relative to the total weight of the composition.

5. The composition as claimed in claim 4, characterized in that the content of nicotinic acid, or of amide of nicotinic acid, is between 1 and 10% by weight relative to the total weight of the composition.

6. The composition as claimed in either one of claims 1 and 2, characterized in that the content of sphingoid base is between 0.01% and 5% relative to the total weight of the composition.

7. The composition as claimed in claim 6, characterized in that the content of sphingoid base is between 0.05% and 2% relative to the total weight of the composition.

8. The composition as claimed in any one of the preceding claims, characterized in that it also comprises ciclopirox or ciclopiroxolamine.

9. The composition as claimed in claim 8, characterized in that the content of ciclopirox or of ciclopiroxolamine is between 0 and 5% by weight relative to the total weight of the composition.

10. The composition as claimed in any one of the preceding claims, characterized in that it is combined with one or more cyclins or with isotretinoin.

11. A cosmetological treatment method, characterized in that it consists in applying to exposed areas a composition based on nicotinic acid or an amide of nicotinic acid, and a sphingoid base, as claimed in any one of claims 1 to 9.

12. The use of nicotinic acid or an amide of nicotinic acid, such as vitamin PP, and of a sphingoid base, for preparing a medicinal product for the treatment of acne and of atopic dermatitis.
